(19) United States
(12) Patent Application Publication
(10) Pub. No.: US 2011/0129850 A1
(43) Pub. Date: Jun. 2, 2011

(54) MICROFLUIDIC PLATFORM FOR CELL CULTURE AND ASSAY

(75) Inventors: Hsian-Rong Tseng, Los Angeles, CA (US); Tak For Yu, Los Angeles, CA (US); Shuling Guo, Los Angeles, CA (US); Owen N. Witte, Sherman Oks, CA (US); Caius Radu, Los Angeles, CA (US); Jenny Shu, Los Angeles, CA (US); Kenichiro Kamei, Los Angeles, CA (US)

(73) Assignee: UCLA Office of Intellectual Property, Los Angeles, CA (US)

(21) Appl. No.: 12/520,376

(22) PCT Filed: Dec. 21, 2007
(86) PCT No.: PCT/US07/26122

§ 371 Date: Jun. 19, 2009

Related U.S. Application Data
(60) Provisional application No. 60/876,525, filed on Dec. 22, 2006.

(51) Int. Cl. G01N 33/53; C12M 3/00; C12M 1/34

(52) U.S. Cl. 435/7.2; 435/289.1; 435/287.1

(57) ABSTRACT

A microfluidic chip for at least one of cell culturing and cell assay has a cell culture chamber defined by the microfluidic chip, a first microchannel defined by the microfluidic chip and constructed to provide a fluid path to said cell culture chamber, the microchannel having a pneumatic valve formed therein to permit selective opening and closing of a fluid path to said cell culture chamber, and a second microchannel defined by the microfluidic chip and constructed to provide a fluid path from the cell culture chamber.

### CROSS-REFERENCE TO RELATED APPLICATION

This application claims priority to U.S. Provisional Application No. 60/876,525 filed Dec. 22, 2006, the entire content of which is hereby incorporated by reference.

### BACKGROUND

1. Field of Invention

The present invention relates to microfluidic devices and methods, and more particularly to microfluidic devices and methods for cell culture and assay.

2. Discussion of Related Art

The development of cell culture and assay for screening potential drug candidates, (J. M. Padron, C. L. van der Wilt, K. Smid, E. Smitskamp-Wilms, H. H. Backus, P. E. Pizao, G. Giaccone, and G. J. Peters, Crit Rev Oncol Hematol, 2000, 36, 141) evaluating biological pathways (L. K. Minor, Curr Opin Drug Discov Devel, 2003, 6, 760) and understanding pharmacological effects (Y. Umezawa, Biosens Bioelectron, 2005, 20, 2504; D. C. Hill, S. K. Wrigley, and L. J. Nisbet, Adv Biochem Eng Biotechnol, 1998, 59, 73) constitute critical technological foundations for a broad spectrum of biomedical research. Conventional cell culture and assay are performed at a macroscopic level, where several constraints, e.g., high sample/reagent consumption, poor precision to control and monitor the microenvironments of cell colonies and the lack of integrated platforms for accurate phenotypic and functional measurements, cause challenges with respect to cost and scalability. Microfluidics, (P. A. Auroux, D. Iossifidis, D. R. Reyes, and A. Manz, Analytical Chemistry, 2002, 74, 2637; D. R. Reyes, D. Iossifidis, P. A. Auroux, and A. Manz, Analytical Chemistry, 2002, 74, 2623; P. S. Dittrich, K. Tachikawa, and A. Manz, Analytical Chemistry, 2006, 78, 3887; P. S. Dittrich and A. Manz, Nat Rev Drug Discov, 2006, 5, 210) with its intrinsic advantages of sample/reagents economy, precise control over physical and chemical microenvironments, high throughput, scalability and digital controllability, provides a prime operation platform for performing biological operations (C. L. Hansen, M. O. A. Sommer, and S. R. Quake, Proceedings of the National Academy of Sciences of the United States of America, 2004, 101, 14431; J. W. Hong, V. Studer, G. Hang, W. F. Anderson, and S. R. Quake, Nature Biotechnology, 2004, 22, 435) and chemical reactions (J. Wang, G. Sui, V. P. Mocharla, R. J. Lin, M. E. Phelps, H. C. Kolb, and H. R. Tseng, Angew Chem Int Ed Engl, 2006, 45, 5276; C. C. Lee, G. Sui, A. Elizarov, C. J. Shu, Y. S. Shin, A. N. Dooley, J. Huang, A. Daridon, P. Wyatt, D. Stout, H. C. Kolb, O. N. Witte, N. Satyamurthy, J. R. Heath, M. E. Phelps, S. R. Quake, and H. R. Tseng, Science, 2005, 310, 1793; J. Wang, Y. L. Bunimovich, G. Sui, S. Savvas, Y. Guo, J. R. Heath, and H. R. Tseng, Chem Commun (Camb), 2006, 3075). Developing integrated and functional microfluidic technology platforms (J. El-Ali, P. K. Sorger, and K. F. Jensen, Nature, 2006, 442, 403; A. Khademhosseini, R. Langer, J. Borenstein, and J. P. Vacanti, Proceedings of the National Academy of Sciences of the United States of America, 2006, 103, 2480) for cell culture and assay can tackle the existing challenges and facilitate contemporary biomedical research.

Numerous examples have been demonstrated for performing cell culture and assay in a variety of microfluidic systems. For example, culture, assay and passage of HeLa cells in a continuos flow microfluidic device have been demonstrated using perfusion channels (P. J. Hung, P. J. Lee, P. Sabounchi, R. Lin, and L. P. Lee, Biotechnol Bioeng, 2005, 89, 1). Based on Braille-driven vales and pump, digitally controlled cell culture and flow-dependent differentiation study of C2C12 myoblast cells have been accomplished (N. Futai, W. Gu, J. W. Song, and S. Takayama, Lab on a Chip, 2006, 6, 149. A micro-culture array with a proper flow resistance arrangement illustrates that an external syringe pump can provide perfusion over a logarithmic range (L. Kim, M. D. Vahey, H. Y. Lee, and J. Voldman, Lab Chip, 2006, 6, 394). A microdevice made of gelatin-based material which mimics an in vivo microenvironment allows cell behavior study in an appropriate manner (A. Paguirigan and D. J. Beebe, Lab Chip, 2006, 6, 407). Challenges remain to perform integrated operations, e.g., parallel cell culture and sequential cell assay of multiple cell lines in a stand-alone microfluidic chip. Therefore, there is a need for improved microfluidic chips for cell culture and/or assay and methods.

### SUMMARY

A microfluidic chip for at least one of cell culturing and cell assay according to an embodiment of the current invention has a cell culture chamber defined by the microfluidic chip, a first microchannel defined by the microfluidic chip and constructed to provide a fluid path to said cell culture chamber, the microchannel having a pneumatic valve formed therein to permit selective opening and closing of a fluid path to said cell culture chamber, and a second microchannel defined by the microfluidic chip and constructed to provide a fluid path from the cell culture chamber.

An incubation box according to an embodiment of the current invention has a plurality of sides defining an enclosed space suitable to receive a microfluidic chip and permitting a plurality of pneumatic fluid lines to access said microfluidic chip when disposed therein to control pneumatic values within microchannels of said microfluidic chip.

A method of performing a biological test according to an embodiment of the current invention includes culturing a plurality of different cell lines in a respective cell culture chamber on a microfluidic chip, and exposing each of the plurality of different cell lines to an environmental stimulus.

A method of performing a biological test according to an embodiment of the current invention includes culturing a plurality of cell lines in a respective cell culture chamber on a microfluidic chip, and exposing each of the plurality of cell lines to a different environmental stimulus.

A method of performing a biological operation according to an embodiment of the current invention includes culturing a plurality of cell lines in a respective cell culture chamber on a microfluidic chip, and performing genetic manipulation on the plurality of cell lines.

### BRIEF DESCRIPTION OF THE DRAWINGS

The invention can be better understood by reading the following detailed description with reference to the accompanying figures in which:

FIG. 1 is a schematic illustration of a portion of a microfluidic chip according to an embodiment of the current invention (a pair of channels provides continuous, open (right) or closed (left) loop, medium feeding);

FIG. 2(a) is a schematic representation of the integrated microfluidic chip according to an embodiment of the current invention for performing cell culture and assay under a digitally controlled interface;

FIG. 2(b) is a photograph of the actual device according to this embodiment of the current invention (it is loaded with various colors of food dyes to enhance the visualization of different parts in the entire system: red and yellow as in part FIG. 2(a); blue indicates the flow channels);

FIG. 3 is a schematic diagram that illustrates the four sequential processes for performing an on-chip cell culture experiment via the cooperation of valves and pumps;

FIG. 4 shows a miniaturized cell incubation box capable of control humidity and pH balance according to an embodiment of the current invention;

FIG. 5 shows cell morphology pictures, cell count, numbers of proliferating cell and floating cell over time;

FIG. 6 shows photographs of parallel cell culture in a stand alone microfluidic chip according to an embodiment of the current invention;

FIG. 7 shows an example of cell culture and sequential apoptotic/living assay in a stand-alone microchip according to an embodiment of the current invention;

FIG. 8 shows bright field and fluorescence micrographs of the cell after the processes of DNA transfection and EGFP driven by a COX-2 promoter induction by TPA in a single microchip according to an embodiment of the current invention;

FIG. 9 shows that hESC can be grown on mEFs in a microfluidic device according to an embodiment of the current invention; and

FIG. 10 shows staining for undifferentiated hESC in a microfluidic device according to an embodiment of the current invention.

### DETAILED DESCRIPTION

In describing embodiments of the present invention illustrated in the drawings, specific terminology is employed for the sake of clarity. However, the invention is not intended to be limited to the specific terminology so selected. It is to be understood that each specific element includes all technical equivalents which operate in a similar manner to accomplish a similar purpose. All cited references are incorporated by reference herein.

Integrated microfluidic systems offer new opportunities for spatial and temporal control of cell culturing by combining surface modifications that mimic in vivo microenvironments (e.g., extracellular matrix) with digitally-controlled microfluidic modules that regulate supply of cell culture media. By further integrating chip-based analytical microfluidic components with the cell culture system, a multi-functional platform for performing complex biological and medical analysis becomes available for facilitating biomedical research.

Cell Culture and Assay

Cell culture. Conventional cell culture techniques have evolved slowly over the last several decades. In this conventional cell culture setting, cell colonies are grown on large surfaces with defined chemical/physical properties (e.g., polystyrene/glass dishes or wells) and in homogeneous culture media. However, in vivo cells respond to spatially and temporally organized signals in the surrounding microenvironment. There is thus a need for new cell culture systems capable of manipulating in vivo microenvironment for cell culture.

Cell culture and assay. Cell assay using living cells, which enables researchers to perform a complex analysis of living systems, is one of the most important methods in the biological fields. For example, a biological assay—an experiment that uses living cells to test the effect of chemicals, is an indispensable technique for drug screening, chemical-safety evaluation and other basic research in life science. The conventional bioassay, however, involves laborious procedures and consumes a significant amount of biological samples and precious reagents.

Micro Total Analysis Systems (μTAS) and Integrated Microfluidics

Micro total analysis systems. In recent years, micro total analysis systems (μTAS) have been of great interest to biological researchers for cellular analysis. A prominent characteristic of μTAS is the capability of constructing highly integrated/functional systems on a microchip. Therefore, many processes that were complicated in conventional cellular analysis could be integrated on stand-alone microchips. This integration resulted in short-time analysis and easy handling for operation. Moreover, these integrated microfluidic systems had advantages such as a reduction in the consumption of cells, reagents, and samples, real-time analysis, and constancy of experimental conditions (Park, T. H. & Shuler, M. L. Integration of cell culture and microfabrication technology. Biotechnol Prog 19, 243-53 (2003)). As stated above, cellular analysis on an integrated microchip can provide numerous benefits. Thus, cellular analysis on microchips has been rapidly spreading, for example, to applications of cell sorting (Fu, A. Y., Chou, H. P., Spence, C., Arnold, F. H. & Quake, S. R. An integrated microfabricated cell sorter. Anal Chem 74, 2451-7 (2002)), and the introduction of genes into cells. However, there have been few papers about the integration of all processes of a bioassay using living cells on a microchip.

Integrated microfluidics. Poly(dimethylsiloxane) (PDMS)-based integrated microfluidics represents a large scale architecture of fluidic channels that allow for the execution and automation of sequential physical, chemical and biological processes on the same device with digital control of operations (Xia, Y. N. & Whitesides, G. M. Soft lithography. Angewandte Chemie-International Edition 551-575 (1998); Quake, S. R. & Scherer, A. From micro- to nanofabrication with soft materials. Science 1536-1540 (2000)). In particular, the elasticity of PDMS materials enable a parallel fabrication of the micron-scale functioning modules, such as valves, pumps and columns (Unger, M. A., Chou, H. P., Thorsen, T., Scherer, A. & Quake, S. R. Monolithic microfabricated valves and pumps by multilayer soft lithography. Science 113-116 (2000)), that are necessary in the sequential operations. In addition, fabrication of intricate devices using this technology requires only relatively simple facilities: the fluidic and control networks are mapped using standard CAD software and transferred onto transparent photomasks. Photolithographic techniques are used to produce a reusable mold onto which a PDMS resin is poured and cured by baking. Access to the fluidic channels is achieved by punching holes through the bulk material, and the devices are readily bonded to glass or silicon substrates, for example. Large arrays of active components, such as valves and pumps, can be created by stacking multiple, individually fabricated layers. When pressurized with air or inert gases, a channel on the control layer that crosses a channel on the flow layer can be deflected, sealing the flow channel and stopping fluid movement. This method of valve operation also constitutes binary switches (e.g., open or closed) of the microfluidics chip. Using this fabrication technology, our joint research team has demonstrated devices of remarkable diversity, including microfluidic devices with chemical reaction circuits (PCT Int. Appl. 2006, WO 2006071470, UCLA case ID No. 2005-280-1), an integrated microfluidic blood sampler for mice (UCLA Case No. 2005-659-1), a high throughput screening platform for high-affinity inhibitors (PCT/US2007/005248, UCLA Case No. 2005-606-1) and a microfluidic platform for high sensitivity quantification of radioisotope concentrations (UCLA Case No. 2006-388-1).

Human Embryonic Stem Cell Culture

Human embryonic stem cells (hESCs). Human embryonic stem cells (hESCs) are pluripotent cells that have the potential to differentiate into all three germ layers and possibly all tissues of the human body (Odorico, J. S., Kaufman, D. S. & Thomson, J. A. Multilineage differentiation from human embryonic stem cell lines. Stein Cells 19, 193-204 (2001)). hESCs were originally isolated from the inner cell mass of human embryos (blastocyst) (Thomson, J. A. et al. Embryonic stem cell lines derived from human blastocysts. Science 282, 1145-7 (1998)) and can be passaged through 100 divisions in vitro. The protocols for differentiation of hESCs have been successfully established in vitro for many cell types (Odorico, J. S., Kaufman, D. S. & Thomson, J. A. Multilineage differentiation from human embryonic stem cell lines. Stem Cells 19, 193-204 (2001); Assady, S. et al. Insulin production by human embryonic stem cells. Diabetes 50, 1691-7 (2001); Kaufman, D. S. & Thomson, J. A. Human ES cells—haematopoiesis and transplantation strategies. J Anat 200, 243-8 (2002); Levenberg, S., Golub, J. S., Amit, M., Itskovitz-Eldor, J. & Langer, R. Endothelial cells derived from human embryonic stem cells. Proc Natl Acad Sci USA 99, 4391-6 (2002); Mummery, C. et al. Cardiomyocyte differentiation of mouse and human embryonic stem cells. J Anat 200, 233-42 (2002); Park, C. H. et al. In vitro and in vivo analyses of human embryonic stem cell-derived dopamine neurons. J Neurochem 92, 1265-76 (2005)). hESCs not only hold considerable promise for the treatment of a number of devastating diseases, but also provide excellent systems for studying early development and human diseases.

Conventional hESC culture. hESCs are conventionally maintained in culture with feeder cells and/or mixtures of exogenous factors. Mouse embryonic fibroblasts (mEFs) are usually used as feeder cells for hESC culture. And also, Matrigel, which is purified from mouse Engelbreth Holm-Swarm tumor, is used for extracellular matrix for hESC culture. Serum is also used for hES cell culture as the source of various growth factors to maintain hES cells.

Xeno-free culture for hESC. The therapeutic potential of ES cells lies in the transplantation of differentiated cell types for disorders such as Parkinson's disease and diabetes which arise from loss, or malfunction, of a single cell type. With a view to future transplantation of hESC derivatives, it is important, therefore, to eliminate or, at least reduce, the potential for contamination by pathogens, etc., from the mouse feeder cells, Matrigel and serum. To eliminate those factors, a large number of combinations of growth factors (GFs) and extracellular matrixes (ECMs) have been screened in search of optimized cell culture environment. However, the screening processes in search of xeno-free culture conditions will consume a lot of GFs and ECM components when a conventional cell culture setting is applied. High cost in sample consumption limited further exploration of this type of research activities.

By incorporation of isolation valves (M. A. Unger, H. P. Chou, T. Thorsen, A. Scherer, and S. R. Quake, Science, 2000, 288, 113) and peristaltic pumps (H. P. Chou, M. A. Unger, and S. R. Quake, Biomed Microdevices, 2001, 3:4, 323), the individual addressability and digital controllability can be conferred into each cell culture chamber, allowing integration of complicated operations in a single device.

This aspect of the current invention can provide new types of PDMS-based integrated microfluidic circuits for performing parallel cell culture and sequential cell assay in an automated fashion. A number of cell lines, including NIH3T3 mouse fibroblast cells, HeLa human epithelial carcinoma cells, B16 mouse melanoma cells and sensitive human embryonic stem cells (HSF1) have been cultured and analyzed in integrated microfluidic circuits according to embodiments of the current invention. We believe that this technology platform has potential to replace conventional cell culture and assay setting with advantages, including sample/reagents economy, high throughput operation, experimental fidelity, scalability, flexibility and digital controllability.

High throughput cell culture and assay. This microfluidic platform has the potential to significantly enhance the throughput of cell analysis by integrating and automating the various cell-handling and cell-processing steps prior to separation and by substantially reducing the separation run times while maintaining high separation efficiencies. While most cellular applications of microfluidics have been directed at analysis of cell contents, it is apparent that such automated, miniaturized instrumentation would also be of use for continuous monitoring of chemical events at living cells. By using a microfluidic device which we have developed, we can culture various kinds of cells in a microfluidic device, and perform various experiments in a device. Additionally, our devices can be easily coupled with high-sensitivity detection instrumentation (e.g., a fluorescent microscope and a CCD camera). Multiple operations have to be integrated on a microfluidic device in order to automate cell culture and assay. Since we have already shown automated chemical reaction systems for sequential production of PET imaging probes (Lee, C. C. et al. Multistep synthesis of a radiolabeled imaging probe using integrated microfluidics. Science 310, 1793-6 (2005)) and parallel screening of high-affinity enzyme inhibitors, we can apply those automation systems for multiple cell culture and assay.

Microenvironment for cells. While microfluidics has shown considerable promise as a tool for studying cell biology, the potential for microfluidics to create more in vivo-like in vitro environments is still largely untapped. It is becoming clear that the scale of the microenvironment provided by microchannels is an important biological parameter. Microchannels have been used for several steps in the in vitro production of embryos typically either matching or improving the performance of previous methods (Aeschlimann, D. & Thomazy, V. Protein crosslinking in assembly and remodelling of extracellular matrices: the role of transglutaminases. Connect Tissue Res 41, 1-27 (2000); Raty, S. et al. Embryonic development in the mouse is enhanced via microchannel culture. Lab Chip 4, 186-90 (2004)). Insect cell cultures as well have shown very different dimension-dependent growth kinetics in microscale cultures as compared to macroscale flask cultures (Yu, H., Meyvantsson, I., Shkel, I. A. & Beebe, D. J. Diffusion dependent cell behavior in microenvironments. Lab Chip 5, 1089-95 (2005)). In our microfluidic device, we found that cells require continuous micro-circulation of culture medium for their proliferation.

Smaller size and less cost. An intrinsic advantage of cell culture in a microfluidic device according to embodiments of the current invention is that we can reduce the volume of medium, growth factors and extracellular matrices, and so on. This means we can reduce the cost for cell culture and assay, too. Especially for hES cell culture, it takes huge cost to identify the best combination of GFs and ECMs. The volume of our microfluidic device can be 1000 times smaller than that of a conventional culture dish.

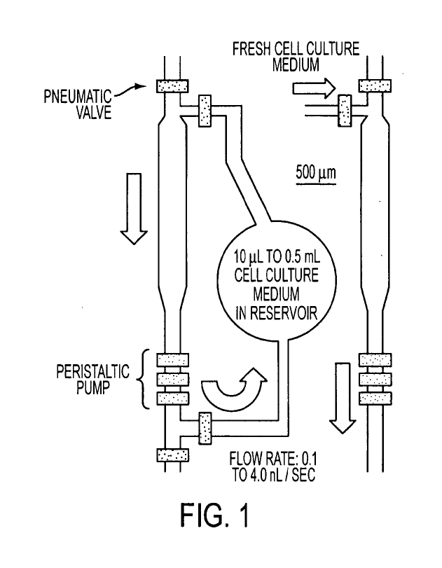

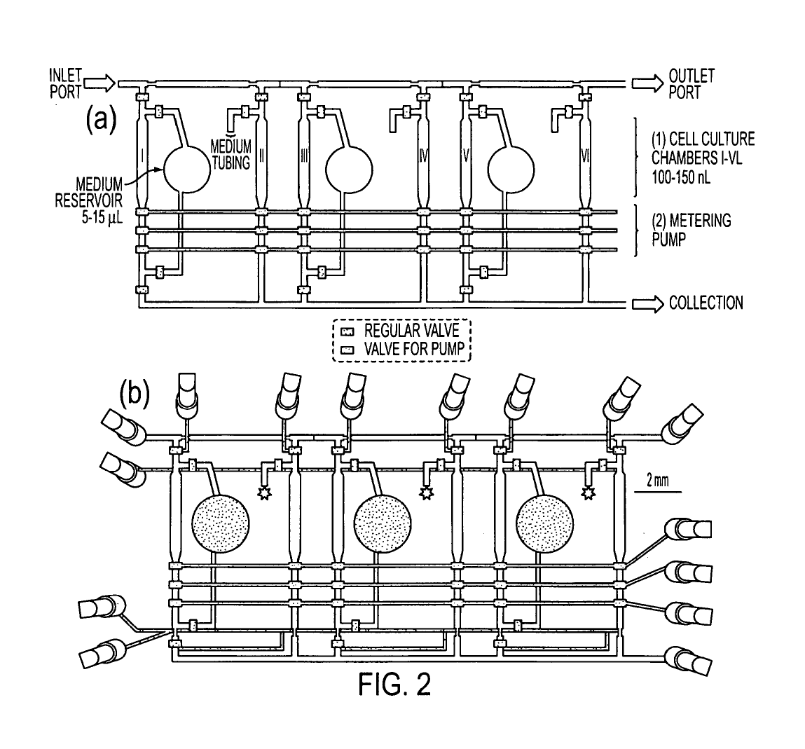

A prototypical cell culture microchip based on the PDMS-based integrated microfluidic system according to an embodiment of the current invention is illustrated in FIG. 1. In other words, FIG. 1 illustrates a portion of a microfluidic chip according to an embodiment of the current invention. FIG. 2 illustrates schematically as well as shows a photograph of a microfluidic chip that has three pairs of cell culture chambers according to an embodiment of the current invention. The three pairs of parallel-oriented cell culture chambers are incorporated in this example, where multiple cell types can be cultured under two different modes of medium supply, i.e., circulatory (channels i, iii and v) and direct feeding (channels ii, iv and vi). The operation of this microchip is controlled by pressure driven valves with their delegated functions indicated by their colors: red for regular valve (for isolation and gating) and yellow for pumping valve (for fluid transportation and circulation). Pneumatic micro-valves and peristaltic micro-pumps were incorporated into the microchips for controlled loading of suspended cell mixture and culture media. Extracellular matrix components (e.g., fibronectin (FN), laminin, Matrigel and RGD peptide) can be coated onto the surfaces of all channels for immobilization of cells. In this case, the dimension of each cell culture chamber is 500 μm (W)×3000 μm (L)×80 μm (H). However, the invention is not limited to only these specific dimensions. External (off-chip) or internal (on-chip) medium reservoir was coupled with the cell channels such that different types of media could be quantitatively delivered to cells in a continuous, open or closed loop respectively. To illustrate parallelization, three pairs of channels were allocated into a single chip. Micro-pumps were connected and therefore synchronized and provided equal flow rates.

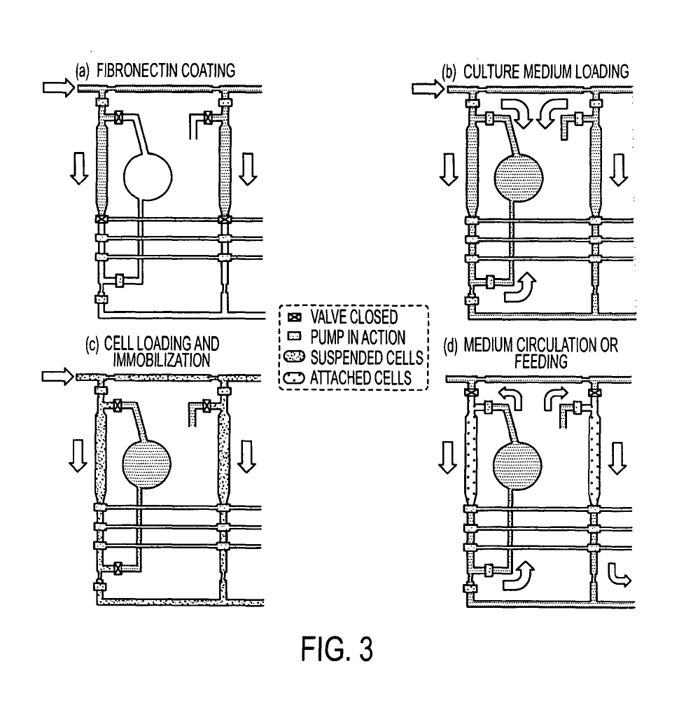

FIG. 3 is a schematic diagram that illustrates the four sequential processes for performing an on-chip cell culture experiment via the cooperation of valves and pumps. In FIG. 3(a) Fibronectin coating: A fibronectin solution (1 mg mL⁻¹) is introduced to fill the cell culture chambers by a dead end filling approach in order to enhance the biocompatibility of the microenvironment. FIG. 3(b) shows culture medium loading: A cell culture medium is loaded to replace the fibronectin solution. Sequentially, the medium reservoir is filled with the culture medium at an external pressure (10-15 psi). FIG. 3(c) shows cell loading and immobilization: A cell suspension solution (1-4×10⁶ cells mL⁻¹) is loaded into the chambers by gravitation, and the microchip is maintained at 37° C. for cell immobilization. FIG. 3(d) shows medium circulation or feeding: The conjugated peristaltic pumps are turned on to circulate medium in the cell culture chamber on the left and to directly feed medium through the one on right. The circulating/feeding flow rates (0.1-4 mL sec⁻¹) are synchronized by the operating frequency of the pumps.

Culturing a Variety of Cell Lines in Microchips.

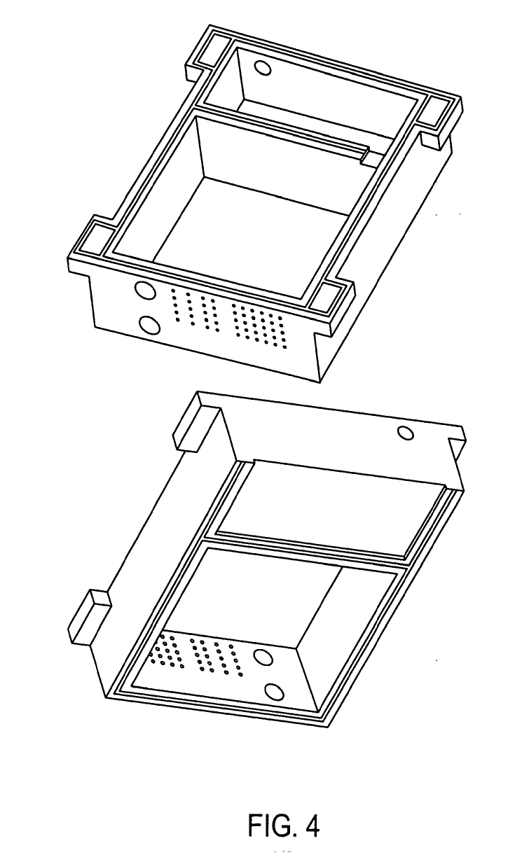

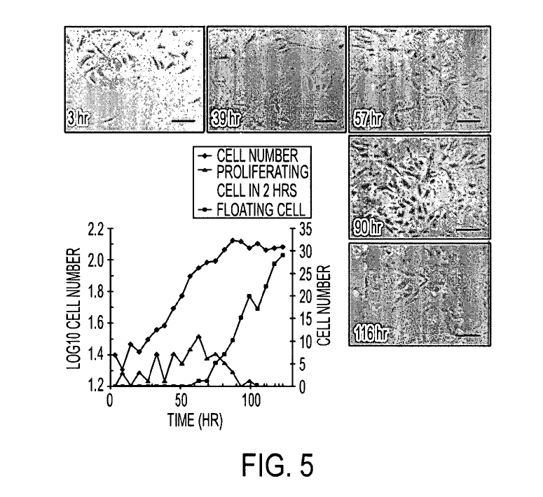

NIH3T3 cell line was first selected for an example according to some embodiments of the current invention. The suspended 3T3 cell mixture obtained from regular cell culture setting was introduced into the fibronectin-coated microchambers which were kept in a custom-designed incubation box to maintain humidity and pH balance (FIG. 4). FIG. 4 shows a miniaturized cell incubation box capable of control humidity and pH balance. This incubation box is made of transparent plastic which allows direct monitoring via a CCD camera in conjunction with a fluorescent microscope. After cells spread on the channel for half an hour, nutrient was flowed through the cell channels. Cell morphology was captured regularly by a CCD-camera over time (FIG. 5). FIG. 5 shows cell morphology pictures, cell count, numbers of proliferating cell and floating cell over time. The cell count and pictures demonstrate a general cell growth behavior. The medium flow was from left to right. The scale bars represent 100 μm. Channels coupled with medium circulation demonstrated healthier cell morphology and proliferation until confluence at 83 hours. This behavior was validated by the triplicate pairs of channels. Biologically, this suggests that the cell endocrine system provides essential signaling molecules in which the medium itself cannot supply, and continuous open systems cannot facilitate.

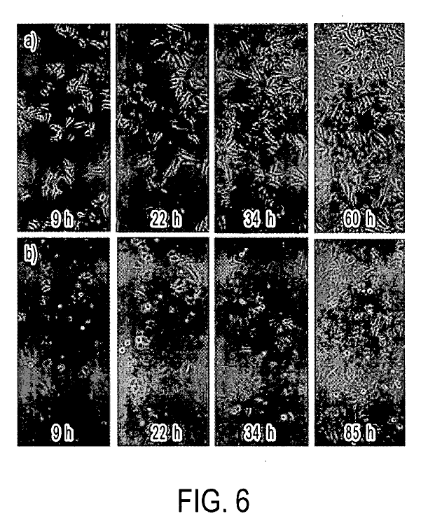

Generally cell number as well as the proliferation rate (as measured by the number of proliferating cells in the FIG. 5) increased with time, except that there were some drops caused by cells walking out of the video window due to cell motility. Like batch cell cultures, the cells went through the standard growth phases: (1) lag: 0-10 hours, (2) exponential: 10-80 hours, and (3) stationary: 80-90 hours. The process of cell proliferation was seen to pass several stages: (1) cell detachment (early M-phase), (2) dividing (late M-phase), and (3) reattachment (G1-phase). The cell took about one hour to finish the proliferation process (M-phase) and then spent most of the time (G1-, S-, and G2-phase), e.g. twenty hours, preparing for the next dividing process. It is intriguing to observe that at high cell density, after cells float off, some cells were delayed to stick back to the channels, whereas some cells either followed the medium flow or halted in the floatation stage without proceeding further (as indicated by the number of floating cell in the FIG. 5). This could be due to the occupancy of channels by cells. Besides 3T3 cells, B16 melanoma cells and HeLa carcinoma cells can also be cultured in microfluidic channels (FIG. 6). FIG. 6 shows photographs of parallel cell culture in a stand alone microfluidic chip according to an embodiment of the current invention. FIG. 6(a) B16 and FIG. 6(b) HeLa show that they can be cultured in a parallel fashion according to an embodiment of the current invention. Parallelization of a number of cell culture chambers constituted a cell array, which could be utilized for performing cell assay in a parallel fashion.

Cell Assay on a Microfluidic Chip.

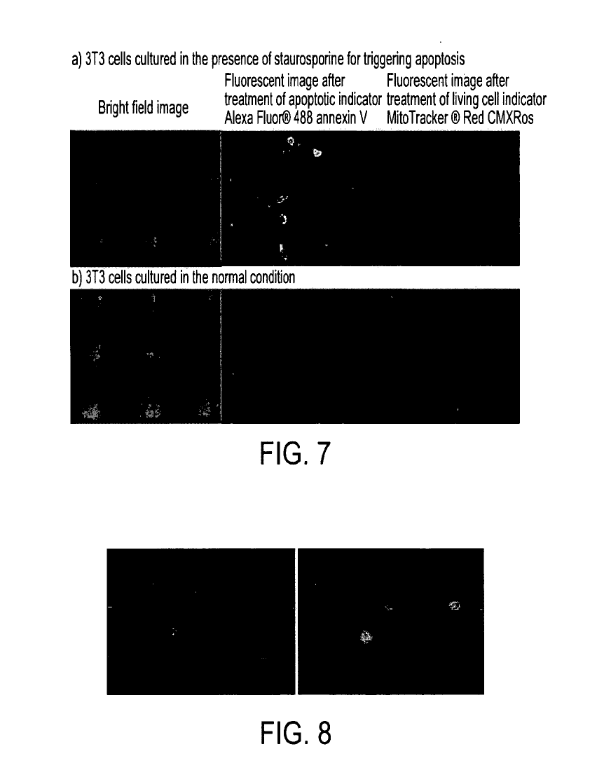

By further integrating chip-based analytical microfluidic components with the chip-based cell culture system, a multi-functional platform for performing complex biological and medical analysis was demonstrated. A cell apoptosis assay was demonstrated using the cell culture/assay chip according to an embodiment of the current invention. Here, an apoptotic stimulant, staurosporine was introduced into a specific cell chamber containing a B16 cell colony. Meanwhile, a negative control experiment was performed in parallel at a normal cell culture condition (without staurosporine). After 2 hours of incubation, fluorescence staining of Alexa Fluor® 488 annexin V and MitoTracker® Red CMXRos dye was performed for indication of apoptotic dead and living cells, respectively. These results are summarized in FIG. 7.

Genetic Manipulation on a Microfluidic Chip.

We have also demonstrated the transfection of 3T3 cells following chip-based cell culture in a single device according to an embodiment of the current invention. This transfection process was performed according to the handbook of SuperFect®

Transfection Reagent from Qiagen. Prior to the transfection processes the 3T3 cells were plated in the microchip for a day. The transfection with the plasmid carrying an enhanced green fluorescent protein (EGFP) driven by a cyclooxygenase-2 (COX-2) promoter and induction through 12-O-tetradecanoylphorbol-13-acetate (TPA) was done within serum-free medium. Fluorescence imaging was then carried out after 6-8 hours from the TPA loading. FIG. 8 shows bright field and fluorescence micrographs of the cell after the processes of DNA transfection and EGFP driven by a COX-2 promoter induction by TPA in a single microchip according to an embodiment of the current invention.

hESC Culture.

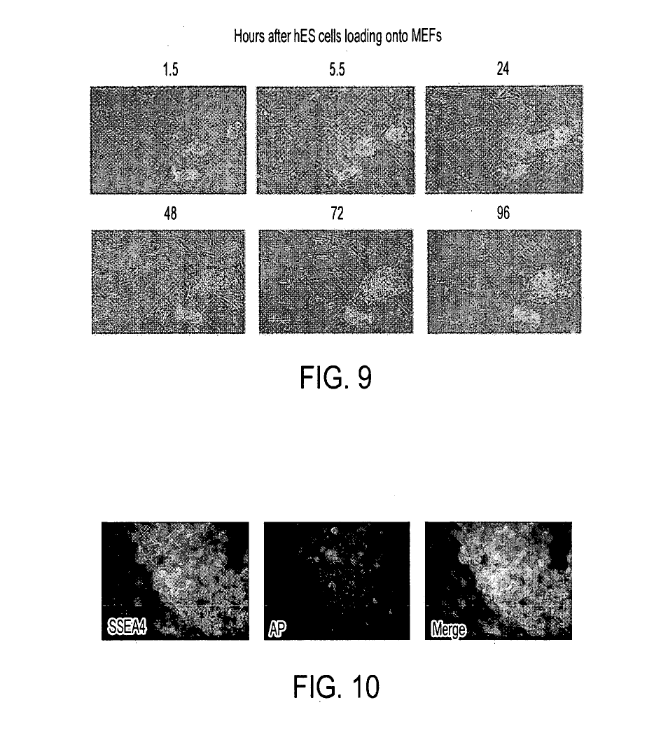

Highly sensitive human embryonic stem cell (hESC) line (HSF1) can also be cultured in the same microfluidic setting according to an embodiment of the current invention. In general, the HSF1 hESCs are cultured on γ-ray-irradiated mouse embryonic fibroblast (MEF) feeder cells in DMEM/F-12 supplied with 20% KnockOut Serum Replacement (GIBCO), 2 mM L-glutamine, 1.1 mM 2-mercaptoethanol, 1 mM nonessential amino acids, and 8 ng/ml bFGF. Cells are passaged at the ratio of 1 to 6-10 every 4-5 days by using 1 mg/ml collagenase type IV and Dispase in DMEM/F12. In our study, the surfaces of the cell incubation chambers in the microfluidic device were coated with fibronectin for 30 min at 37° C. Then 0.1% gelatin solution was loaded into the chamber and incubated for 12 hours at 37° C. Irradiated MEF feeder cells were loaded into a device, and cultured for 24 hours at 37° C. hESCs were loaded into a device, and cultured for a week. Medium for hESC was routinely changed every day. As the result, hESC can be cultured and grown in a microfluidic device for 6 days (FIG. 9).

Staining for undifferentiated hESCs. Sequential cell assays were performed following cell culture for identification of undifferentiated hESCs. The chip-cultured hESCs were fixed by 2% formaldehyde, and stained by alkaline phosphatase (AP) activity. Undifferentiated hESC was also stained through immunocytochemistry. The antibodies used were: mouse anti-SSEA4 (stage specific embryonic antigen 4) at 1:10 as primary antibody, and anti-mouse IgG conjugated with FITC. The cells cultured in a microfluidic device for 6 days were positive for both AP staining and immunocytochemistry (FIG. 10). hESC can be cultured in our microfluidic device with keeping the undifferentiated stage. FIG. 10 shows staining for undifferentiated hESC in a microfluidic device according to an embodiment of the current invention ((A) Immunostaining for SSEA-4 (B) Alkaline phosphatase (AP) staining (C) Merge with (A) and (B)).

The embodiments illustrated and discussed in this specification are intended only to teach those skilled in the art the best way known to the inventors to make and use the invention. Nothing in this specification should be considered as limiting the scope of the present invention. The above-described embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

We claim:

1. A microfluidic chip for at least one of cell culturing and cell assay, comprising:
a cell culture chamber defined by the microfluidic chip;
a first microchannel defined by the microfluidic chip and constructed to provide a fluid path to said cell culture chamber, said microchannel having a pneumatic valve formed therein to permit selective opening and closing of a fluid path to said cell culture chamber; and
a second microchannel defined by the microfluidic chip and constructed to provide a fluid path from said cell culture chamber.

2. A microfluidic chip according to claim 1, wherein said microfluidic chip is constructed of a size and material composition suitable to be used with an optical microscope.

3. A microfluidic chip for at least one of cell culturing and cell assay, comprising:
a first plurality of cell culture chambers defined by said microfluidic chip;
a plurality of cell culture medium reservoirs defined by said microfluidic chip, each being arranged in a one-to-one correspondence with a respective one of said first plurality of cell culture chambers; and
a second plurality of cell culture chambers defined by said microfluidic chip, each being arranged in a one-to-one correspondence with a respective one of said first plurality of cell culture chambers and a respective one of said plurality of cell culture medium reservoirs.

4. A microfluidic chip according to claim 3, wherein said microfluidic chip is constructed of a size and material composition suitable to be used with an optical microscope.

5. A microfluidic chip according to claim 3, wherein at least one of said plurality of cell culture chambers is constructed to be self-circulating during operation and another of said plurality of cell culture chambers is constructed to have a flow-through connection with external fluid sources and sinks during operation.

6. An incubation box, comprising:
a plurality of sides defining an enclosed space suitable to receive a microfluidic chip and permitting a plurality of pneumatic fluid lines to access said microfluidic chip when disposed therein to control pneumatic values within microchannels of said microfluidic chip.

7. An incubation box according to claim 6, wherein said incubation box is constructed of a size and material composition suitable to be used with an optical microscope.

8. A method of performing a biological test, comprising:
culturing a plurality of different cell lines in a respective cell culture chamber on a microfluidic chip; and
exposing each of said plurality of different cell lines to an environmental stimulus.

9. A method of performing a biological test, comprising:
culturing a plurality of cell lines in a respective cell culture chamber on a microfluidic chip; and
exposing each of said plurality of cell lines to a different environmental stimulus.

10. A method of performing a biological operation, comprising:
culturing a plurality of cell lines in a respective cell culture chamber on a microfluidic chip; and
performing genetic manipulation on said plurality of cell lines.
